(19) United States
(12) Patent Application Publication
(10) Pub. No.: US 2010/0256476 A1
(43) Pub. Date: Oct. 7, 2010

(54) TRACHEAL TUBE LOCATING SYSTEM AND METHOD

(75) Inventor: Lockett E. Wood, Lyons, CO (US)

Correspondence Address: NELLCOR PURITAN BENNETT LLC;ATTN: IP LEGAL, 6135 Gunbarrel Avenue, Boulder, CO, 80301, US

(73) Assignee: Nellcor Puritan Bennett LLC, Boulder, CO (US)

(21) Appl. No.: 12/418,699

(22) Filed: Apr. 6, 2009

(51) Int. Cl. A61B 5/05; A61M 16/04

(52) U.S. Cl. 600/409; 128/207.14

(57) ABSTRACT

According to various embodiments, a tracheal tube may include a detector array configured to detect a field generated by an external source. The detector array may generate a signal indicative of a distance between the source and a distal end of the tracheal tube. Based on a known distance between the source and a carina, a distance between the distal end of the tracheal tube and the carina may be computed. The distance information may provide an indication as to whether the tracheal tube is properly placed within the trachea.

### BACKGROUND OF THE INVENTION

The present disclosure relates generally to medical devices and, more particularly, to airway devices, such as tracheal tubes.

This section is intended to introduce the reader to various aspects of art that may be related to various aspects of the present invention, which are described and/or claimed below. This discussion is believed to be helpful in providing the reader with background information to facilitate a better understanding of the various aspects of the present invention. Accordingly, it should be understood that these statements are to be read in this light, and not as admissions of prior art.

In the course of treating a patient, a tube or other medical device may be used to control the flow of air, food, fluids, or other substances into the patient. For example, tracheal tubes may be used to control the flow of air or other gases through a patient's trachea. Such tracheal tubes may include endotracheal (ET) tubes, tracheotomy tubes, or transtracheal tubes. In many instances, it is desirable to provide a seal between the outside of the tube or device and the interior of the passage in which the tube or device is inserted. In this way, substances can only flow through the passage via the tube or other medical device, allowing a medical practitioner to maintain control over the type and amount of substances flowing into and out of the patient.

For example, a patient may be intubated when an endotracheal tube is inserted through the patient's mouth and into the trachea. Often, such intubation procedures may be performed during medical emergencies or during critical care situations. As such, healthcare providers may balance a desire for speed of intubation with a desire for accurate placement of the tube within the trachea. However, proper placement of a tracheal tube may be complex. In certain situations, placement may be aided with visualization of the trachea performed during laryngoscopy. During an intubation procedure, a practitioner may employ a lighted laryngoscope during introduction of the endotracheal tube. However, often the visualization of the trachea is poor because of patient secretions that may obscure the laryngoscope. In addition, such visualization during introduction of the tube may not account for ongoing changes in the tube position within the trachea that may occur when a patient coughs, which may dislodge a tube from its desired location, or when a patient is jostled or moved within a care setting, which may change the position or angle of the tube within the trachea.

### SUMMARY

Certain aspects commensurate in scope with the originally claimed invention are set forth below. It should be understood that these aspects are presented merely to provide the reader with a brief summary of certain forms of the invention might take and that these aspects are not intended to limit the scope of the invention. Indeed, the invention may encompass a variety of aspects that may not be set forth below.

Some embodiments described herein are directed to a system for determining placement of a tracheal tube in a subject. The system may include a field source positionable outside of the subject at a location corresponding to an anticipated location of the tracheal tube and configured to emanate a field into the subject. The system may also include a tracheal tube configured to be disposed in the trachea of the subject and at least one field detector disposed on or in the tracheal tube and configured to generate a signal based upon detection of the field. Furthermore, the system may include a monitor coupled to the at least one field detector and configured to provide an indication to a human user of a position of the tracheal tube in the subject.

Other embodiments described herein are directed to a tracheal tube that may include a detector array disposed within the tracheal tube and including multiple detectors each capable of detecting a field, such as a magnetic or electromagnetic field. The tracheal tube may also include a connector communicatively coupled to the detector array and configured to interface with an external device capable of determining tracheal tube position within a subject based on a signal from the detector array.

Further embodiments described herein are directed to a method for determining placement of a tracheal tube in a subject. The method may include disposing the tracheal tube in a trachea of the subject, the tracheal tube including at least one field detector disposed in or on the tracheal tube. The method may also include disposing a field source at a location corresponding to an anticipated location of the tracheal tube and configured to emanate a field into the subject. Furthermore, the method may include detecting a signal from the at least one field detector and determining a position of the tracheal tube in the subject based upon the signal.

### BRIEF DESCRIPTION OF THE DRAWINGS

Advantages of the invention may become apparent upon reading the following detailed description and upon reference to the drawings in which:

FIG. 1 is a schematic diagram of a system including a tracheal tube with field detectors configured to detect the position of the tracheal tube relative to a field source in accordance with an embodiment;

FIG. 2 is a detailed schematic diagram of the field detectors of FIG. 1 in accordance with an embodiment;

FIG. 3 is a flow diagram of a method of operating a tracheal tube in accordance with an embodiment;

FIG. 4 is a perspective view of the tracheal tube of FIG. 1 in accordance with an embodiment; and

FIG. 5 is a cross-sectional side view of a distal end of the tracheal tube of FIG. 1 showing the position of the field detectors relative to the field source in accordance with an embodiment.

### DETAILED DESCRIPTION OF SPECIFIC EMBODIMENTS

One or more specific embodiments of the present invention will be described below. In an effort to provide a concise description of these embodiments, not all features of an actual implementation are described in the specification. It should be appreciated that in the development of any such actual implementation, as in any engineering or design project numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

A tracheal tube may be used to seal a patient airway and provide positive pressure to the lungs when properly inserted into a patient trachea. Positioning the tracheal tube at a desired position within the trachea, for example during endotracheal intubation, may improve the performance of the tracheal tube and reduce clinical complications. In particular, the distal inserted end of the endotracheal tube may be positioned in the patient trachea at a location substantially between the vocal cords and carina. If a tube cuff is not inserted far enough past the vocal cords, for example the tube may become more easily dislodged. If the tube is inserted too far into the trachea, such as past the carina, then the tube may only function to adequately ventilate one of the lungs, rather than both. Thus, proper placement of the distal tip of the tube may result in improved ventilation to the patient.

Provided herein are tracheal tubes and systems for facilitating proper placement of the tracheal tube relative to certain anatomical structures in and around the patient airway and trachea. Such tracheal tubes include one or more field detectors positioned along the length of the tube. The field detectors may measure the field strength from a field source positioned adjacent to an external anatomical feature of a patient (e.g., suprastemal notch). The field detectors may be communicatively coupled to an external monitor configured to determine a position of a distal end of the tracheal tube relative to the field source/external anatomical feature based on a signal from the field detectors. In certain embodiments, this position may be utilized to compute a distance from the distal end of the tracheal tube to the carina (or other internal anatomical structure or tissue), based upon a known distance of one or more sensors that produce output signals (and the relative strength of the signals if more than one sensor is used) and the end of the tube. A healthcare provider may then use the information about the location of the tracheal tube relative to the anatomical structures (e.g., the carina) to determine whether the tube is properly placed or whether the position of the tube should be adjusted.

In certain embodiments, the disclosed tracheal tubes, systems, and methods may be used in conjunction with any appropriate medical device, including without limitation a feeding tube, an endotracheal tube, a tracheotomy tube, a circuit an airway accessory, a connector, an adapter, a filter, a humidifier, a nebulizer, nasal cannula, or a supraglottic mask/tube. The present techniques may also be used to monitor any patient benefiting from mechanical ventilation, e.g., positive pressure ventilation. Further, the devices and techniques provided herein may be used to monitor a human patient, such as a trauma victim, an intubated patient a patient with a tracheotomy, an anesthetized patient, a cardiac arrest victim, a patient suffering from airway obstruction, or a patient suffering from respiratory failure.

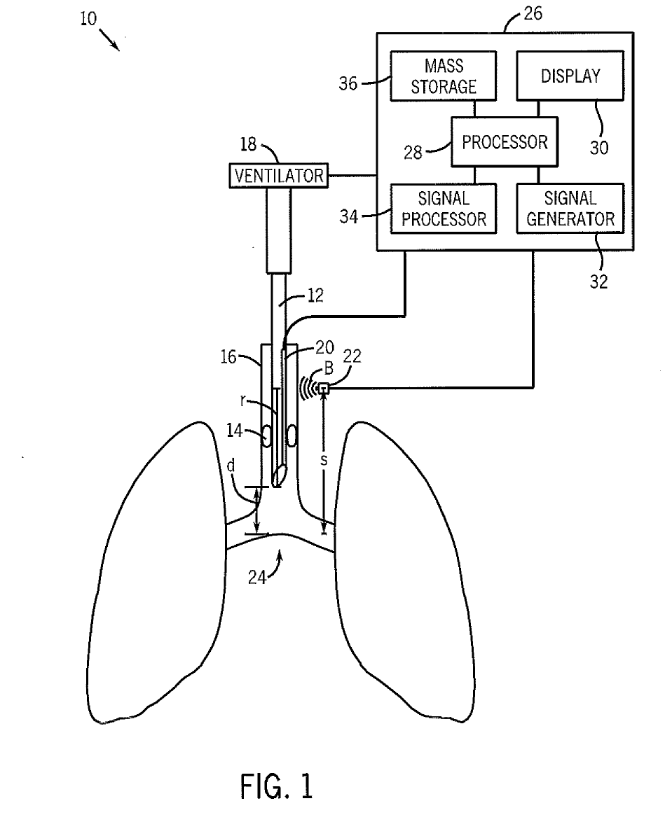

FIG. 1 is a schematic diagram of a tracheal tube system 10 that has been inserted into a patient trachea. The system 10 includes a tracheal tube 12, shown here as an endotracheal tube, with an inflatable balloon cuff 14 that may be inflated to form a seal against tracheal walls 16. When ventilation is provided via the tube system 10, a ventilator 18 is typically provided, as discussed below. As illustrated, the tracheal tube 12 includes a field detector array 20 configured to detect a field B from an external field source 22. The field detector array 20 may include a single field detector or multiple field detectors arranged along the length of the tracheal tube 12. As discussed in detail below, the field detector array 20 may generate a signal indicative of detection of the field B emanating from the field source 22. A distance r between the detected location of the field source 22 and a distal end of the tracheal tube 12 may be computed based upon the known distance of the detector or detectors generating signals indicative of detection of the field source and the tube end. Moreover, a physician or technician may know or estimate the distance between the location of the field source (e.g., at the suprasternal notch) and an anatomical feature of interest (e.g., a carina 24), typically based upon the size of the patient. Thus, based upon these known distances, a distance d between the distal end of the tracheal tube 12 and the carina 24 may be computed. In this manner, the tracheal tube 12 may be properly positioned. It should be noted that, as described below, the location of the detector or detectors along the tracheal tube 12 (and particularly their distance from the tube end) will be known, although the distance or distances from the tube end may differ for different sizes or configurations of tracheal tubes. The system 10 may make the calculations described herein with such knowledge, based upon user input of tube data, automatic acquisition of tube data, or a combination of the two.

When the system 10 includes devices that facilitate positive pressure ventilation of a patient, such as ventilator 18, any ventilator may be used, such as those available from Nellcor Puritan Bennett LLC. The system 10 also includes a monitor 26 that may be configured to implement embodiments of the present disclosure. The monitor 26 may be a stand-alone device or may be coupled to another patient monitor or to the ventilator 18. The monitor 26 may include a processor 28 and a display 30. The processor 28, or any other suitable processing circuitry, aids in computing the distance d of the distal end of the tube 12 from reference structures within the patient, such as the carina 24.

The monitor 26 may include certain elements for controlling the field source 22 and/or receiving signals from the detector array 20. For example, in certain embodiments, the field source 22 may be an electromagnet and the detector array 20 includes magnetic sensors. In such configurations, a signal generator 32 within the monitor 26 may provide the field source 22 with a direct current (DC) or alternating current (AC) electrical signal. Field source 22 may convert this electrical signal into a magnetic field B. The detector array 20 may then generate an output signal representative of detection of the field B. This signal may be received by the monitor 26 and analyzed by a signal processor 34. Depending upon the location of the detector, or if more than one detector is utilized, the distance between the detector generating the strongest signal, the signal processor 34 may convert the magnetic field detection signal into an electrical signal indicative of the distance r from the field source 22 to the distal end of the tracheal tube 12. The processed signal may then be conveyed to the processor 28 where the position d is computed. In alternative embodiments, the field source 22 may be configured to emit a radio frequency (RF) signal and the detector array 20 may include RF receivers (e.g., antennas). In such embodiments, the signal generator 32 and the signal processor 34 may function in a similar manner to the previously described embodiment with regard to generating and analyzing signals, respectively. Similarly, the field source 22 may include a permanent magnet. In all cases, moreover, the field source 22 may be temporarily positioned in the desired location (e.g., as a hand-held probe), or may be semi-permanently secured to the patient, such as via adhesive or tape.

The monitor 26 may be configured to receive signals from the detector array 20 and store the signals in a mass storage device 36, such as RAM, PROM, optical read/write storage devices, flash memory devices, hardware storage devices, magnetic storage devices, or any other suitable device permitting memory storage. The signals may be accessed and operated upon according to instructions (which may also be stored in the memory circuitry) executed by the processor 28. In certain embodiments, the signals may be related to a placement of the tracheal tube 12 within the trachea and may be processed by the monitor 26 to indicate whether the tracheal tube 12 is properly placed. The monitor 26 may be configured to provide an indication about the placement of the tracheal tube 12 within the trachea, such as an audio alarm, visual alarm or a display message, as well as to provide special signals in the event the tracheal tube 12 is too far or too close to certain anatomical structures, such as the carina 24, or outside of a predetermined placement range, or whether the tube has moved or moved more than an allowed amount since its initial placement.

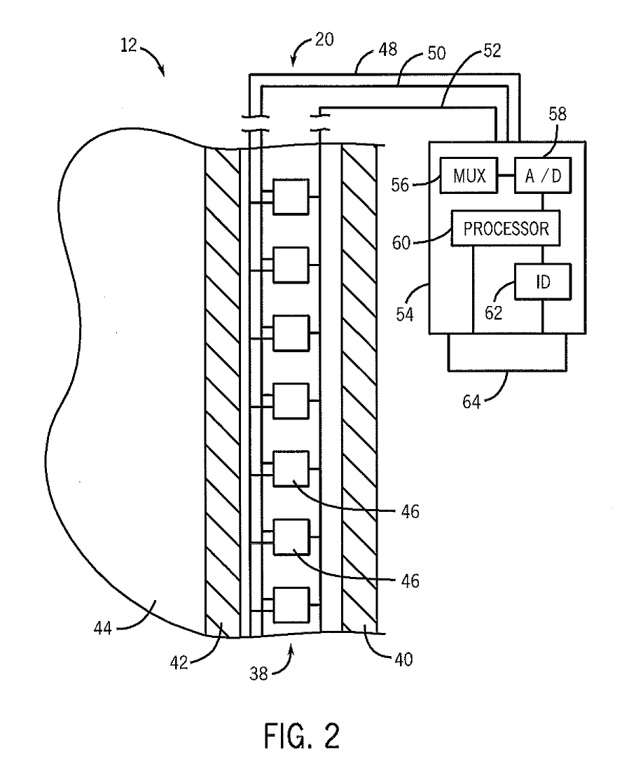

FIG. 2 is a detailed schematic diagram of the field detector array 20 shown in FIG. 1. In certain embodiments, the detector array 20 may be embedded within the tracheal tube 12. Specifically, the tracheal tube 12 may include a lumen 38 formed between an outer wall 40 and an inner wall 42 of the tracheal tube 12. The inner wall 42 defines a gas flow passage 44 configured to provide pressurized air to lungs. The lumen 38 may include a substantially circular passage that extends along the length of the tracheal tube 12. In certain embodiments, the lumen 38 is approximately 1 mm in diameter. Alternative embodiments may include lumens 38 having larger or smaller diameters, such as 0.25 mm, 0.5 mm, 0.75 mm, 1.25 mm, 1.5 mm, 1.75 mm, 2 mm, or larger. A plurality of such lumens may be provided for different purposes, one of which being available for housing the detector array 20 in the present technique. Therefore, a diameter of the field detector array 20 may be less than the diameter of the lumen 38. In alternative embodiments, the field detector array 20 may be embedded within the tube wall 40. For example, the field detector array 20 may be inserted within the tube wall 40 as the tracheal tube 12 is formed by an extrusion process. In further embodiments, the field detector array 20 may be disposed to an outside surface of the outer wall 40 or an inside surface of the inner wall 42 by an adhesive connection, for example.

In certain presently contemplated embodiments, the field detector array 20 includes multiple field detectors 46 arranged in a linear configuration along the length of the tracheal tube 12. While seven detectors 46 are illustrated in the figure, more or fewer field detectors 46 may be employed. For example, certain embodiments may include 1, 2, 4, 6, 8, 10, 15, 20, 25, 30, 40, 50, or more field detectors 46. As discussed in detail below, this configuration may facilitate determination of the distance r between the field source 22 and the distal end of the tracheal tube 12. For example, the field source 22 may be an electromagnet or a permanent magnet and the detectors 46 may be magnetic detectors. A variety of magnetic field detectors may be employed, such as Hall-effect transducers, giant magnetoresistance (GNM) sensors, and magnetostrictive linear position sensors, for example. Hall-effect transducers are illustrated in FIG. 2 and may be well-suited for disposition within the tracheal tube 12 because such transducers may only minimally impact magnetic resonance imaging (MRI) scan quality.

According to Hall-effect principles, a voltage may be generated perpendicular to both a magnetic field and a direction of current flow through the sensor 46. This voltage may be proportional to a magnitude of the magnetic field. Therefore, by passing a current through the sensors 46, a magnetic field magnitude may be measured by determining the voltage generated perpendicular to both the magnetic field and the direction of current flow.

As illustrated, in this embodiment three conductors are electrically coupled to each Hall-effect transducer via a supply bus 48, a neutral bus 50 and an output bus 52. Current is supplied to the sensors 46 via the supply bus 48 and the neutral bus 50. Based on the magnetic field magnitude, a voltage is returned via the output bus 52 and the neutral bus 50. While the output bus 52 is represented as a single conductor, it should be appreciated that one output conductor for each sensor 46 may be included within the output bus 52. Furthermore, other bus configurations may be employed in alternative embodiments. For example, the sensors 46 may be configured to output a digital signal indicative of field intensity. Each bus 48, 50 and 52 is electrically coupled to a connector 54. The connector 54 is configured to communicatively couple the detector array 20 to the monitor 26.

In one presently contemplated embodiment the connector 54 includes a multiplexer CRUX) 56, an analog to digital converter (A/D) 58, a processor 60 and an identification storage device (ID) 62. The elements 56, 58, 60 and 62 may be individual electrical components or constituents of a single component. A voltage from each Hall-effect transducer 46 may pass through the output bus 52 to the multiplexer 56. The multiplexer 56 is configured to convert the individual signals from each sensor 46 into a single analog signal. The A/D converter 58 converts the analog signal to a digital signal indicative of the signal generated by each sensor 46. The processor 60 may analyze the digital signal to compute the position of the field source 22 relative to the distal end of the tracheal tube 12. As previously described, the output signal produced by each Hall-effect transducer 46 is proportional to the strength of the detected magnetic field. Because the magnetic field flux density decreases as distance from the source 22 increases, sensors 46 closer to the source 22 may produce a stronger signal (e.g., higher voltage) than sensors 46 farther from the source 22. As a result, the processor 60 may determine the position of the tracheal tube 12 relative to the source 22 by comparing output signals of the sensors 46 along the tube 12.

The connector 54 may also include an identification storage device 62. The device 62 may include information regarding the geometric configuration of the tracheal tube 12. For example, tracheal tubes 12 may be selected based on patient size. In other words, longer tracheal tubes 12 may be selected for taller patients, while shorter tracheal tubes 12 may be selected for shorter or smaller patients. Therefore, determination of proper tube position may be dependent on the selected tracheal tube 12. Consequently, the identification storage device 62 may include information indicative of tube length. This information may be used to determine the distance d between the distal end of the tracheal tube 12 and the carina 24, for example. In addition, the information may also be conveyed to the monitor 26. In alternative embodiments, tube identification information may be encoded on a barcode attached to the tracheal tube 12 and/or within a code printed on the tube that may be entered into the monitor 26. The data may also be stored in a memory (e.g., in the form of a lookup table or database) in the monitor 26 or even remote from the monitor 26 (e.g., accessed by a network link). In a straightforward implementation, for example, a single (or few) field detectors 46 may be used, and the output signals may be conveyed directly to the monitor 26, which may process the signals to determine the position of the tracheal tube 12 with respect to the field source 22, and output a visual or audible signal (e.g., illuminate a light or produce a sound) indicating the position of the tube 12. Similarly, where different tubes having different geometries and detector placements are used, the tube identifying information or detector placement may be input manually by a user.

The connector 54 may be coupled to the monitor 26 or other device (e.g., computer) via a port 64. The port 64 may employ any standard protocol such as USB or I²C, or a proprietary communication standard. In addition, electrical power may be provided to components within the connector 54 and/or the supply bus 48 via the port 64. For example, if the port 64 is a USB port, the monitor 26 may provide 5V power to the connector 54 and/or detector array 20, as established by the USB standard.

Alternative detector array 20 and/or connector 54 configurations may be employed in alternative embodiments. For example, in certain embodiments, the source 22 may be an RF transmitter and the sensors 46 may be antennas configured to detect RF radiation. Such configurations may function in a substantially similar manner to the embodiment described above with regard to the magnetic source 22 and magnetic sensors 46. Specifically, the magnitude of an RF signal may decrease as distance from the source 22 increases. Therefore, the antennas 46 closer to the source 22 may receive a stronger signal than the antennas 46 farther from the source 22. This magnitude difference may be utilized to compute the tracheal tube 12 position just as magnetic field magnitude differences were utilized in the previously described embodiment.

In addition, the source 22 may be configured to emit an AC or DC signal. A DC signal may be characterized by a generally static magnetic or electromagnetic field. As previously described, such fields may be detected by the detector array 20 for determination of tracheal tube 12 position. Alternative embodiments may employ a field source 22 that creates a field based upon an AC signal characterized by a time-varying amplitude. Similar to the DC signal, the detected magnitude of the resulting field may be utilized to determine tracheal tube 12 position. However, the AC signal may provide enhanced reception. For example, the source 22 may be configured to emit an AC signal at a particular frequency. The processor 60, processor 28 and/or the signal processor 34 may be configured to filter all frequencies detected by the detectors 46 except a frequency of interest emitted by the source 22. In this manner, external interference may be reduced, thereby providing enhanced signal reception.

Furthermore, different connector 54 configurations may be employed in alternative embodiments. For example, in certain embodiments, the connector 54 may not include any of the above described electronic circuits (i.e., components 56, 58, 60 and 62). In such configurations, the connector 54 may pass analog signals from the detector array 20 directly to the monitor 26. The monitor 26 may include circuits configured to determine tracheal tube 12 position based on the signals from each sensor 46. In further embodiments, the connector 54 may only include the identification storage device 62 to identify the tracheal tube 12 to the monitor 26.

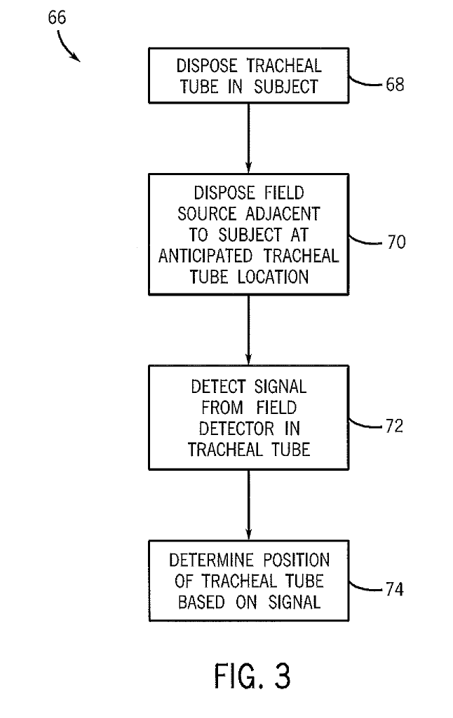

FIG. 3 is a process flow diagram illustrating an exemplary method in accordance with some embodiments. The method is generally indicated by reference number 66 and includes various steps or actions represented by blocks. First, as represented by block 68, the tracheal tube 12 is disposed in a patient or subject. As previously discussed, this step may involve inserting the tube 12 into the trachea. Next, the field source 22 is disposed adjacent to the patient at an anticipated tracheal tube 12 location, as represented by block 70. For example, the field source 22 may be placed against a suprasternal notch of the patient. Because a distance between the suprasternal notch and the carina 24 may be known or estimated based on the size of the patient (e.g., height), the suprasternal notch may be a welt-suited location for placing the field source 22, although other locations may be selected. As represented by block 72, a signal from the field source 22 is detected by the field detector array 20 within the tracheal tube 12. Finally, the position of the tracheal tube 12 is determined based on the signal, as represented by block 74. As noted above, this may include determination of the distance r between the field source 22 and the distal end of the tracheal tube 12 Based on the known distance s between the source 22 and the carina 24, the distance d between the distal end of the tracheal tube 12 and the carina 24 may be calculated. In certain embodiments, an audio and/or visual alarm may be activated if the distance d deviates from a predetermined range. These steps may be performed during intubation or at any stage thereafter (e.g., after the cuff 14 has been inflated and the tube 12 is anticipated to remain relatively stationary).

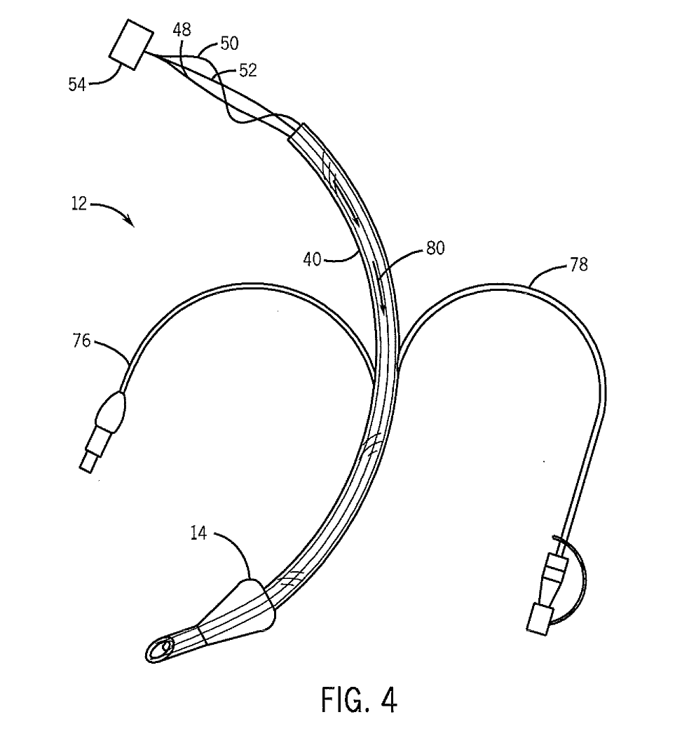

FIG. 4 is a perspective view of a tracheal tube 12 according to certain embodiments of the present techniques. As shown, the tube 12 includes a cuff 14 that may be inflated via inflation lumen 76. The tracheal tube 12 also includes a suction lumen 78 for aspirating secretions that may form above the cuff 14. As previously discussed, the tracheal tube 12 includes busses 48, 50 and 52. As shown, the busses 48, 50 and 52 may extend through the walls 40 of the tracheal tube 12 such that they are substantially in line with a flow path 80 of the tracheal tube 12. The busses 48, 50 and 52 are coupled to the connector 54 that may interface with the monitor 26.

The tube 12 and the cuff 14 may be formed from materials having suitable mechanical properties (such as puncture resistance, pin hole resistance, tensile strength), and chemical properties (such as biocompatibility). In one embodiment, the walls of the cuff 14 are made of a polyurethane having suitable mechanical and chemical properties. An example of a suitable polyurethane is Dow Pellethane® 2363-80A. In another embodiment, the walls of the cuff 14 are made of a suitable polyvinyl chloride (PVC). In one embodiment, the cuff 14 may be generally sized and shaped as a high volume, low pressure cuff that may be designed to be inflated to pressures between about 15 cm H₂O and 30 cm H₂O. The system 10 may also include a respiratory circuit (not shown) connected to the endotracheal tube 12 that allows one-way flow of expired gases away from the patient and one-way flow of inspired gases toward the patient. The respiratory circuit, including the tube 12, may include standard medical tubing made from suitable materials such as polyurethane, polyvinyl chloride (PVC), polyethylene teraphthalate (PETP), low-density polyethylene (LDPE), polypropylene, silicone, neoprene, polytetrafluoroethylene (PTFE), or polyisoprene.

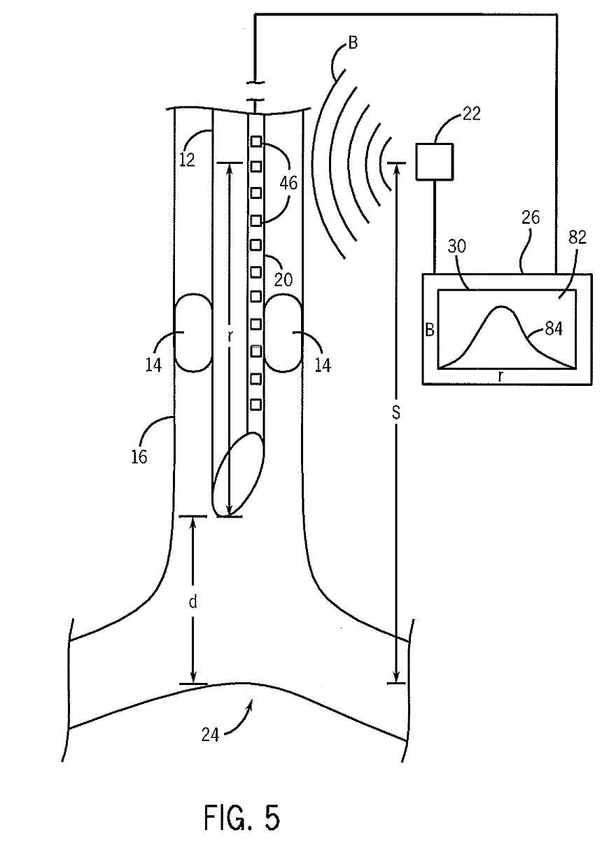

FIG. 5 is a cross-sectional side view of the distal end of the tracheal tube 12, showing the position of the field detectors 46 relative to the field source 22. As previously discussed, the field source 22 may be placed adjacent to an external anatomical feature of the patient having a known distance from the carina 24. For example, the field source 22 may be placed on the suprasternal notch. The distance from the suprasternal notch to the carina 24 may be known or estimated based on the size of the patient. Therefore, by placing the source 22 on the suprasternal notch, the distance s may be a known value.

In addition, the distance r between the distal end of the tracheal tube 12 and the source 22 may be determined by measuring the magnitude of the field B emitted by the source 22 across the detector array 20. For example, if the source 22 emits a magnetic field B and the sensors 46 are Hall-effect transducers configured to provide a signal proportional to the magnetic field B, the distance r may be computed by analyzing the voltages produced by the sensors 46. As illustrated, the detector array 20 is communicatively coupled to the monitor 26. The monitor 26 may be configured to convert the signals from each sensor 46 into a magnetic field magnitude. Because the distance between each sensor 46 and the distal end of the tracheal tube 12 is known (e.g., encoded within the identification storage device 62), the magnetic field along the tube 12 may be computed. For example, the monitor 26 includes a display 30 configured to provide a graph 82 of magnetic field magnitude B as a function of distance r from the distal end of the tracheal tube 12. As seen by the shape of curve 84, magnetic field magnitude B is inversely proportional to distance from the sensor 46. Therefore, the peak of curve 84 represents the position of the closest sensor 46 to the source 22. In other words, the distance r is determined by comparing the distance from the distal end of the tracheal tube 12 to the maximum magnetic field magnitude.

Once the distance r is determined, the distance d between the distal end of the tracheal tube 12 and the carina 24 may be calculated by subtracting distance r from distance s. In this manner, the tube 12 may be properly placed within the trachea. For example, proper tube placement may involve adjusting tube insertion depth to achieve a distance d between approximately 1 cm to 5 cm. In certain embodiments, the monitor 26 may automatically compute the distance d and display a value indicative of distance d on the display 30. For example, the display 30 may express the distance d in terms of inches or millimeters between the distal end of the tube 12 and the carina 24. In alternative embodiments, the display 30 may display a graphical representation of a trachea, including the carina 24 and the tracheal tube 12. In this manner, a clinician may utilize the image on the monitor 26 to determine proper tube placement.

Further embodiments may include an audible and/or visual alarm that is activated if the distance d varies from a predetermined range, or if the tube 12 is detected to move more than a desired amount from a known or initial position. It should be appreciated that there may be several empirically derived target ranges, depending on the size, age, or gender of the patient. A target range to which the distance d may be compared may differ for adult men, who may have, in an embodiment, a target range of about 3-4 cm, and adult women, for whom the target range may be about 2-3 cm. The alarm may indicate to a clinician that the distance d has deviated from the target range. In other embodiments, the alarm may be triggered if the distance d is less than 3 cm, less than 2 cm, or less than 1 cm.

While the disclosure may be susceptible to various modifications and alternative forms, specific embodiments have been shown by way of example in the drawings and have been described in detail herein. However, it should be understood that the embodiments provided herein are not intended to be limited to the particular forms disclosed. Indeed, the disclosed embodiments may not only be applied to measurements of tracheal tube placement relative to anatomical structures in the trachea, but these techniques may also be utilized for the measurement and/or analysis of the placement of other suitable medical devices relative to other anatomical structures. For example, the present techniques may be utilized for the measurement and/or analysis of tracheal tubes relative to tracheal walls or the vocal cords. In addition, the present techniques may be employed in determining appropriate placement of any medical device, such as a stent, catheter, implant, feeding tube, cardiac device, drug delivery device, or pump. Rather, the various embodiments may cover all modifications, equivalents, and alternatives falling within the spirit and scope of the disclosure as defined by the following appended claims.

What is claimed is:

1. A system for determining placement of a tracheal tube in a subject comprising:
a field source positionable outside of the subject at a location corresponding to an anticipated location of the tracheal tube and configured to emanate a field into the subject;
a tracheal tube configured to be disposed in the trachea of the subject;
at least one field detector disposed on or in the tracheal tube and configured to generate a signal based upon detection of the field; and
a monitor coupled to the at least one field detector and configured to provide an indication to a human user of a position of the tracheal tube in the subject.

2. The system of claim 1, wherein the field source is configured to emit a magnetic field and the at least one field detector is configured to detect the magnetic field.

3. The system of claim 2, wherein the field source comprises a permanent magnet, an electromagnet, or a combination thereof.

4. The system of claim 2, wherein the at least one field detector comprises a Hall-effect transducer, a giant magnetoresistance sensor, or a magnetostrictive linear position sensor.

5. The system of claim 1, wherein the field source is configured to emit a radio frequency signal and the at least one field detector is configured to detect the radio frequency signal.

6. The system of claim 1, comprising a plurality of field detectors disposed along the tracheal tube and coupled to the monitor.

7. The system of claim 6, wherein the monitor is configured to provide an indication of the position of the tracheal tube based upon relative signal strength from the plurality of field detectors.

8. The system of claim 1, wherein the monitor is configured to display a representation of the position of the tracheal tube in the subject based upon the signal.

9. A tracheal tube, comprising:
a detector array disposed within the tracheal tube and including a plurality of detectors each capable of detecting a magnetic or electromagnetic field; and
a connector communicatively coupled to the detector array and configured to interface with an external device capable of determining tracheal tube position within a subject based on a signal from the detector array.

10. The tracheal tube of claim 9, wherein the detector array is disposed within a wall of the tracheal tube.

11. The tracheal tube of claim 10, wherein the detector array is disposed within a lumen in the wall.

12. The tracheal tube of claim 9, wherein the connector includes a power bus configured to provide electrical power to the detector array.

13. The tracheal tube of claim 9, wherein the connector includes a signal bus configured to convey the signal from the detector array to the external device.

14. The tracheal tube of claim 9, wherein the connector comprises a processor capable of determining tracheal tube position within the subject based on the signal from the detector array.

15. The tracheal tube of claim 9, comprising means for identifying the tracheal tube to the external device.

16. A method for determining placement of a tracheal tube in a subject comprising:
disposing the tracheal tube in a trachea of the subject, the tracheal tube including at least one field detector disposed in or on the tracheal tube;
disposing a field source at a location corresponding to an anticipated location of the tracheal tube and configured to emanate a field into the subject;
detecting a signal from the at least one field detector; and
determining a position of the tracheal tube in the subject based upon the signal.

17. The method of claim 16, comprising determining a distance between the at least one field detector on or in the tracheal tube and a distal end of the tracheal tube, and wherein determining the position of the tracheal tube includes determining a distance of the distal end from an anatomical feature of interest.

18. The method of claim 16, comprising emitting an alarm if the position of the tracheal tube in the subject deviates from a predetermined range.

19. The method of claim 16, comprising identifying the tracheal tube from among a plurality of candidate tracheal tubes to determine a relative location of the at least one field detector with respect to at least one other feature of the tracheal tube, and wherein determining the position of the tracheal tube in the subject includes referring to the relative location based upon the identification of the tracheal tube.

20. The method of claim 19, wherein the at least one field detector is coupled to a cable and connector assembly, and wherein the tracheal tube is identified by data stored in the cable and connector assembly.
